(19) United States
(12) Patent Application Publication
(10) Pub. No.: US 2023/0420123 A1
(43) Pub. Date: Dec. 28, 2023

(54) CONTROL FOR AN EXTRACORPOREAL CIRCULATORY SUPPORT

(71) Applicants: Hochschule Offenburg, Offenburg (DE); Xenios AG, Heilbronn (DE)

(72) Inventors: Matthias Heinke, Rudolstadt (DE); Johannes Hörth, Offenburg (DE); Steffen Eschinger, Frankfurt (DE); Ivo Simundic, Wendlingen (DE)

(21) Appl. No.: 18/007,438

(22) PCT Filed: Aug. 3, 2021
(86) PCT No.: PCT/EP2021/071695

§ 371 Date: May 30, 2023

(30) Foreign Application Priority Data
Aug. 3, 2020 (DE) 10 2020 004 698.3

(51) Int. Cl. G16H 40/63; A61B 5/369

(52) U.S. Cl. CPC G16H 40/63; A61B 5/369

(57) ABSTRACT

The present disclosure relates to control units for an extracorporeal circulatory support as well as systems comprising such a control unit and corresponding methods. Accordingly, a control unit for an extracorporeal circulatory support is suggested, which is configured to receive a measurement of an ECG signal of a supported patient over a predefined period of time, wherein the ECG signal comprises multiple data points for each time point within a cardiac cycle. The control unit comprises an evaluation unit, which is configured to evaluate the data points for at least one time point spatially and/or temporally and to determine at least one amplitude change within the cardiac cycle from the evaluated data points. The control unit is further configured to output a control signal for an extracorporeal circulatory support at a predefined time point after the at least one amplitude change.

### CROSS-REFERENCE TO RELATED APPLICATIONS

The present application is the national stage entry of International Patent Application No. PCT/EP2021/071695, filed on Aug. 3, 2021, and claims priority to application Ser. No. 10/202,0004698.3, filed in the Federal Republic of Germany on Aug. 3, 2020, the disclosures of which are expressly incorporated herein in their entirety by reference thereto.

### TECHNICAL FIELD

The present disclosure relates to control units for extracorporeal circulatory support as well as systems comprising such a control unit and corresponding methods.

### BACKGROUND

If the pumping capacity or pumping function of the heart fails, a cardiogenic shock may occur, which, due to a reduction in cardiac output or ejection, may result in a reduced perfusion or blood flow through the end organs such as the brain, kidneys, and the vascular system in general. Such acute heart failure results in an acute blood deficiency in the tissues and organs and thus an oxygen deficiency, also called hypoxia, which can lead to damage to the end organs. In most cases, such cardiogenic shock occurs as a result of a complication of acute myocardial infarction (AMI) or heart infarction. However, such life-threatening situations can also occur as a complication of surgical treatment, such as a bypass, or due to inadequate or impaired lung function, and can ultimately result from disorders of the conduction system, structural heart disease, or inflammatory processes of the myocardium. Although factors such as early revascularization, the administration of inotropic drugs, and mechanical support can improve the physiological condition of the patient, the mortality rate in the event of cardiogenic shock remains above fifty percent.

To stabilize the patient's condition, circulatory support systems have been developed that provide mechanical support and can be quickly connected to the circulatory system. They can improve the blood flow and perfusion of the organs, including the coronary arteries of the heart, and avoid a hypoxic state. For example, a blood pump may be connected to a venous access by means of a venous cannula and to an arterial access by means of an arterial cannula for aspiration and forwarding the blood, respectively, to provide a flow of blood from a side with a low pressure, for example via an oxygenator, to a side with a higher pressure and thus support the patient's circulation.

However, the complexity and dynamics of the patient's own heart action require precise timing or coordination of the extracorporeal support. For example, the blood flow in the heart's own coronary arteries, which normally provide the heart muscle with sufficient oxygen, generally occurs in the diastole of the cardiac cycle—thus ensuring that the left ventricle has been emptied accordingly. This is because if the filling pressure in the left ventricle is as low as possible, i.e., at the end of systole or at the beginning of diastole, the coronary arteries can unfold their lumen to the maximum possible extent, thus increasing the blood flow rate and oxygen supply. Accordingly, extracorporeal circulatory support for perfusion of the coronary arteries should be controlled in such a way that perfusion is preferably performed at the beginning of the diastole, whereas perfusion during the systole should be avoided.

To control the extracorporeal support, measurement signals from an electrocardiogram (ECG) can be detected and used to determine corresponding characteristic amplitudes for different phases of the cardiac cycle. For example, an R-peak or R-wave being characteristic of the systolic phase of the cardiac cycle is usually easily distinguishable from other phases of the cardiac cycle, for example in a QRS complex. The R-wave can thus be used, with a given latency, to control a blood pump in a successive diastolic phase.

For the provision of an ECG signal, different ECG leads can be provided, which are positioned at or inserted into different anatomical regions. This causes a certain variability of the measurement signal. Furthermore, stimulation-related or pathophysiological interferences can considerably worsen the ratio of the useful signal to the interfering signal and thus render it difficult to determine the amplitudes in the cardiac cycle, so that the desired amplitude may not be detected or cannot be determined. This not only results in inconsistency in the monitoring of cardiac action and performance. Rather, the control of the extracorporeal circulatory support, which uses the amplitude as a trigger signal, may be triggered at the wrong time, so that support is not provided in the planned cardiac cycle phase.

From DE 10 2010 024 965 A1 a method for determining an R-wave in an ECG signal is known, which improves a synchronization of the ECG signal with an MRI imaging method. The R-wave is determined for a predefined time period by means of a temporal derivative of the ECG signal rather than using a threshold comparison. The derivative of the ECG signal is based on single data points per time point and is subjected to a plausibility test, which takes interferences and variations into account that are specifically caused by the magnetic field. However, no pathophysiological-related or single, spontaneous anomalies and, in particular, no stimulation-related interferences that are caused by an implanted pacemaker are taken into account. The method furthermore always provides a direct synchronization with the R-wave, i.e., without a predefined latency that is essential for the control of an extracorporeal circulatory support.

Accordingly, there is a need to optimize the ratio of the useful signal to the interfering signal from an ECG in such a way that the stability of the trigger signal for the control of an extracorporeal circulatory support is improved under various physiological conditions.

### SUMMARY

In some aspects, the present methods, systems, and devices provide an improved stability of a trigger signal for an extracorporeal circulatory support.

Accordingly, a control unit for an extracorporeal circulatory support is suggested, which is configured to receive a measurement of an ECG signal of a supported patient over a predefined period of time, wherein the ECG signal comprises multiple data points for each time point within a cardiac cycle. The control unit comprises an evaluation unit which is configured to evaluate the data points spatially and/or temporally for at least one time point and to determine at least one amplitude change within the cardiac cycle from the evaluated data points. Furthermore, the control unit is configured to output a control signal for the extracorporeal circulatory support at a predefined time point after the at least one amplitude change.

Different or various cardiac cycles or cardiac actions can be recorded in the predefined time period, wherein each cardiac cycle can define specified time points, for example, from the beginning of the cardiac cycle to the end of the cardiac cycle. This simplifies the comparison between different cardiac cycles, for example, compared to an evaluation using absolute time points. Thus, the different cardiac cycle phases of successive cardiac cycles and especially the course of these cardiac cycle phases of successive cardiac cycles can be compared. Hence, data points are collected at identical points in time in successive cardiac cycles, so that the data points collected at identical points in time in successive cardiac cycles can be compared or offset against each other. Thus a useful signal can be displayed for each point in time of a cardiac cycle phase.

Furthermore, for the same point in time of a single cardiac cycle, different ECG leads can be provided to provide the ECG signal, so that a corresponding number of data points can be provided for each point in time. The different measurement signals thus allow that selective data points from specific ECG leads may be used for processing.

Accordingly, at least two data points are available for each point in time within the predefined time period. Depending on the number of recorded or detected cardiac cycles and/or the number of available ECG leads, more data points may be provided for each point in time. The predefined time period can be defined, for example, by the duration of treatment or by the predefined number of recorded cardiac cycles.

The spatial and/or temporal evaluation hence enables a correction of individual interfering signals such that the determination of at least one amplitude change within the cardiac cycle is facilitated and the accuracy is improved. In other words, such improvement of the useful signal is enabled based on the present at least two data points for each time point and without the need of a reference data set of the ECG signal. Such reference set is anyway not existing or even possible in case of an extracorporeal circulatory support and/or a cardiac stimulation of the patient provided by a pacemaker.

Furthermore, the use of the data points for each point in time enables the determination of the amplitude change in real time, so that, for example, stimulation-related interferences by an implanted pacemaker, pathophysiologically caused interferences or also individual, spontaneous anomalies can be taken into account and these do not complicate the determination of the amplitude change. Thereby, exogenous interfering signals that are not related to the cardiac stimulation are preferably not considered. In particular, exogenous interfering signals, interfering signals that are induced by imaging methods, e.g., induced during MRI imaging or other magnetic field induced interfering signals, are preferably excluded. The direct processing of the ECG signals that have been detected in real-time enables that the control signal is based on the currently measured measurement signals and that the currently received ECG signal is directly, i.e., in particular without temporal delay, taken into account for the circulatory support of the patient. This is in contrast with methods that provide a prognosis of the ECG signal, which is merely based on previously recorded data (i.e., the data are initially collected, stored, and subsequently processed, but not directly used) or which is only used for virtual simulations. Furthermore, the amplitude change at a particular point in time can be provided or expected, so that by means of the data points for at least one point in time it may be monitored whether an amplitude change actually occurs at a given time.

Furthermore, the output of the control signal or regulation signal for the extracorporeal circulatory support may cause an immediate setting or adjustment of a corresponding parameter or operating parameter of a coupled extracorporeal circulatory support device. For example, one or more pump drives or pump heads for blood pumps, e.g., non-occlusive blood pumps, in an extracorporeal circulatory support system may be controlled or regulated. Thus, the ECG signal can be used to provide a desired blood flow rate in a corresponding cardiac cycle phase.

The blood pump can be connected to a venous access via a venous cannula and to an arterial access via an arterial cannula for sucking or pumping the blood to provide a blood flow from a side with a low pressure to a side with a higher pressure. Preferably, the blood pump is formed as a disposable or single-use item and is fluidically separated from the respective pump drive and can be easily coupled, e.g., via magnetic coupling. The control unit actuates the motor of the pump drive by outputting the corresponding signal and can hence cause a change in the speed or revolution speed of the blood pump.

The ECG signal may furthermore be fed into or received from the control unit via an interface that is connected to at least one ECG device. However, the control unit is preferably formed as part of an ECG device or is formed such that the ECG device can be attached to the control unit. Thereby, the control unit can be used independently of other components and may have a compact design. Preferably, the ECG device is integrated in a single housing of a system for extracorporeal circulatory support, for example in the sensor box in the form of an ECG card or an ECG module. Alternatively, however, the control unit may also be configured to receive an external ECG signal from the supported patient, for example from a cardiac monitor arranged outside of an extracorporeal circulatory support system. This allows the system to be made even more compact.

The at least one amplitude change is furthermore preferably a characteristic ECG signal, which enables the control unit to be synchronized with the blood pump, so that a regular or periodic output of the control signal from the control unit may be provided. For example, the change in amplitude or the respective range in the electrical excitation conduction system can be characteristic of the systolic or diastolic phase of the heart, so that a control signal can be output in such a way that a blood pump can be operated at a predefined time and in a predefined phase, without overlapping with other phases.

Preferably, the evaluation unit is configured to evaluate the data points for a predetermined or predefined time interval, based on at least one cardiac cycle phase of the ECG signal, and to determine the at least one amplitude change within the time interval. For example, the data points or the ECG signal may be used to detect or determine a QRS complex so that the at least one amplitude change corresponds to one or more characteristic features.

Restricting the evaluation of the data points to a particular time interval not only facilitates data processing and acceleration of processing, for example, to ensure that the amplitude change may be determined in real time under different conditions, for example, in case of a larger number of data points. It also enables a higher accuracy of the determined amplitude change. For example, amplitude changes that are irrelevant for control can be ignored or hidden and computational capacity can be used for specific data points or one or more points in time and corresponding cardiac cycle phases. At the same time a high resolution of the amplitude change determination is provided.

In order to be able to determine not only an absolute amplitude change for a specific point in time within the cardiac cycle, but also a more precise course of the amplitude, the evaluation unit is preferably configured to determine the at least one amplitude change based on data points for at least two time points. For example, the ECG signal and the corresponding data points may be sampled at a frequency of 500 Hz, so that 2 ms lie between two respective points in time per second. In order to determine a course of the amplitude change or a relative slope, two points in time, either successive points in time or points in time at a distance from each other, may already be sufficient.

Preferably, however, the at least one amplitude change is determined for a larger number of time points of between 2 and 500 time points, further preferably between 50 and 150 or of at least 50 or 100 or 150 time points. For example, the evaluation unit may be configured to determine the at least one amplitude change by evaluating all data points within a QRS complex. Accordingly, the number of time points can be selected depending on the existing cardiac arrhythmias and/or cardiac stimulations. For example, 5 to 10 time points may be selected in case of an increased occurrence of ventricular extrasystoles and 10 to 100 time points in case of irregular and/or rare ventricular and/or supraventricular extrasystoles. Furthermore, the number of time points can also be selected according to the duration of the examination and/or depending on the setup configurations, so that a higher number than 500 points of time may also be provided or selected. The number of time points may also be between 10 and 10,000 time points, for example, in the case of pacemaker dependency and ventricular VVI pacing.

Furthermore, the cardiac output can be provided by a patient's own cardiac action as well as with stimulation, for example with a pacemaker. In these cases, pathophysiological or stimulation-related interferences may occur, which can be suppressed by a specific selection of time points, for example by providing a corresponding time interval for determining the at least one amplitude change.

The provision of a control signal for the extracorporeal circulatory support should, as described in the above, occur at a time and in a physiological state in order to provide maximum support for the cardiac performance. Accordingly, an amplitude change should be determined, which can be used as a temporally stable trigger signal. Therefore, the evaluation unit is preferably configured to determine at least one selected amplitude change, which is characteristic for a cardiac cycle phase. Further preferred is to determine at least one selected amplitude change that is characteristic for a P-wave or, in particular, an R-wave.

However, other amplitude changes can also be determined, for example, over a predefined section of the ECG signal or from a distinctive point of the ECG signal. Preferably, however, at least one R-wave or R-wave is determined from the data points, by means of which a trigger signal with a predefined latency time is output. For example, a control signal for an operating parameter of a blood pump may be output at a predefined time point after the detection of the R-wave, for example, the detection of the maximum amplitude, and the blood pump may be adjusted or set accordingly, typically with a delay.

Accordingly, determining the at least one amplitude change provides a temporally stable, electrocardiographically triggered and hemodynamically optimized synchronized extracorporeal circulatory support.

The evaluation of the data points may be performed both spatially and temporally. Accordingly, the ECG signal preferably comprises at least a first measurement signal from a first ECG lead and a second measurement signal from a second ECG lead, wherein the first and second ECG leads are spatially separated from each other and wherein the evaluation unit is configured to evaluate the data points spatially and to determine the at least one amplitude change based on an addition or averaging of the measurement signals.

The spatial separation of the leads and the corresponding signals, wherein, for example, a spatial and/or anatomical spacing may be present, can ensure, on the one hand, that the distance of the useful signal to particular interfering signals, for example from a stimulation of the heart, is improved and that these interfering signals can thus be avoided as far as possible and be at least partially filtered out, such that they do not impair the determination of the at least one amplitude change. On the other hand, the spatial separation of the ECG leads renders it possible to record or detect an ECG signal with the strongest possible useful signal, even in case of variations or alterations of physiological signals, for example, of the cardiac excitation lines.

The addition or summation or averaging of the measurement signals or the respective, spatially separated data points as part of signal averaging thus improves the ratio of the useful signal to the interfering signal by a factor of at least 1.2, for example 1.4, through the use of multiple ECG leads or signal sources, so that at least one amplitude change can be clearly or unambiguously determined, even in the case of weaker measurement signals or fluctuations. In other words, the ratio of the useful signal to the interfering signal can be improved by a factor of the square root of n for a number of n ECG leads, so that at least one amplitude change can be unambiguously determined even with weaker measurement signals or fluctuations. With two ECG leads an improvement of √(n=2)≈1.41 can be achieved.

This improvement can be achieved, for example, in the presence of ideal noise with all frequencies, but can be reduced in the case of non-ideal noise signals, which can occur, for example, in the case of biosignal interferences.

A corresponding improvement in the ratio of the useful signal to the interfering signal can also be achieved with temporal averaging or signal averaging, wherein the improvement results from the square root number n of averaged heart actions or heart cycles, for example, from at least two averaged R-wave-triggered heart actions. Accordingly, the control signal may be output as a trigger signal with high temporal stability.

Preferably, the ECG signal comprises a measurement signal of a transthoracic ECG lead and/or a transesophageal ECG lead. The number of ECG leads is not limited to the number of the respective data points, so that in principle there is a choice of ECG leads for the evaluation of the data points. For example, a plurality of transthoracic ECG leads (I, II, III, aVR, aVL, aVF, V1, V2, V3, V4, V5, V6) and (bipolar) transesophageal ECG leads (Oeso 12, Oeso 34, Oeso 56, Oeso 78) may be provided for electrographic analysis, wherein one or two of the respective ECG lead types can be used for the data points.

The trigger stability is usually relevant for an entire treatment period and may therefore preferably be monitored over the predefined time period. Accordingly, the evaluation unit may be configured to determine an amplitude change for at least two cardiac cycles and a time interval and/or a frequency of the amplitude changes, wherein the control unit is particularly configured to output a signal characterizing the time interval and/or the frequency.

The characterizing signal can be, for example, a current time interval between the instantaneous or current amplitude change and the last determined amplitude change, for example an R-R interval, and/or for example an average time interval, otentially with a current deviation. The signal can also, for example, cause a graphic representation on a display, wherein the specific or particular amplitude changes in the respective cardiac cycles are marked or identified. Thus, it is possible to determine not only whether the amplitude change was determined at the same or a similar time, but also whether it was determined at the right time, for example, at a maximum value and not at the beginning or end of an amplitude. Accordingly, the temporal stability can be easily visually monitored using the markings.

The evaluation unit is preferably configured to determine the at least one amplitude change continuously for or during each successive cardiac cycle that is detected or recorded from the ECG signal. Thereby, any instability of the trigger signal can be immediately detected and eliminated by adjusting the evaluation. For example, an alternative ECG lead for providing the measurement signal and/or an alternative time interval for evaluating the data points may be selected, wherein the evaluation unit can be advantageously configured to enable an automatic adjusting of a setting in order to provide an improvement with respect to the determined amplitude change. For example, threshold values for the data points or the acquired measurement signals can be stored, optionally with respect to one or more time points, wherein, for example, an alternative ECG lead or an alternative time interval is automatically selected for determining the amplitude change, when a respective threshold value is fallen below or exceeded.

A temporal evaluation of the data points may be provided, additionally or alternatively to the spatial evaluation. Accordingly, the evaluation unit may be configured to evaluate the respective data points of each cardiac cycle, in particular the data points corresponding to each other in time (i.e., those data points of successive cardiac cycles which are each equally spaced in time to a reference point, e.g., the maximum of a signal in the cardiac cycle, in the cardiac cycle) and to determine the at least one amplitude change based on averaging or addition of the corresponding data points recorded for at least one time point from at least two cardiac cycles.

As described in the above, different cardiac cycles or cardiac actions can be recorded in the predefined time period, wherein each cardiac cycle can define points in time, for example from a beginning of the cardiac cycle to the end of the cardiac cycle. Thus, the different cardiac cycle phases of successive cardiac cycles and in particular the course of these cardiac cycle phases can be compared with each other, so that data points for the same time point of different successive cardiac cycles, which are each equally spaced in time (with respect to a defined reference point of the cardiac cycle), represent a useful signal for the same respective cardiac cycle phase. Preferably, the data acquisition is hence performed in such a way that the data points for each cardiac cycle are determined at the same time point before or after a defined reference point, which is selected identically for all cardiac cycles, in the respective cardiac cycle, wherein the reference point is preferably morphologically and/or physiologically predefined. The predefined reference point is typically a characteristic property in the ECG signal, i.e., the onset of one of the ECG signals (P, Q, R, 5, T) or the time point of the maximum of one of these signals, as they occur in each cardiac cycle. For example, in every cardiac cycle the time point of the maximum of the R-wave can be defined as a reference point. The occurrence of this reference point, which is determined by the cardiac cycle, varies from cardiac cycle to cardiac cycle in time, so that, e.g., the R-wave or another characteristic of the cardiac cycle can occur earlier or later in one cardiac cycle than in the following cardiac cycle. Nevertheless, this characteristic of the ECG remains the reference point in each of the cardiac cycles recorded or detected. The measurement of the data points per cardiac cycle is therefore carried out individually for each cardiac cycle, but with constant temporal dependence on this predefined reference point. In each cardiac cycle data points are measured before and after the reference point according to a predefined frequency over the course of the cardiac cycle, i.e. a data point is acquired in each of the acquired cardiac cycles at a constant rate per μs or per 2 μs before and after the occurrence of the reference point.

The temporal averaging, i.e., averaging, or addition of the data points thus render it possible that individual outliers, which, e.g., do not lie in a relevant cardiac cycle range and are therefore not characteristic of a certain cardiac cycle phase, nevertheless do not impair the determination of the amplitude change, in particular since the value of the corresponding data point is relatively small for other cardiac cycles. In this way it can be monitored in real time whether the determined amplitude change is within the predefined range and whether a trigger signal is stable.

Although averaging or addition with data points from two cardiac cycles already improves the useful signal compared to the interfering signal, averaging or addition over more than two cardiac cycles is preferred. Accordingly, the evaluation unit can be configured to determine at least one amplitude change based on averaging or addition of data points from at least 10, e.g., 10 to 100 or 10 to 40 or 10 to 35 cardiac cycles, preferably at least 40, e.g., between 40 and 80 cardiac cycles. As described in the above, an (theoretical) improvement of the useful signal for a number of n cardiac cycles or cardiac actions can be increased by a factor corresponding to the square root, i.e., Vn. With an averaging of 25 cardiac cycles, the useful signal or the interfering signal distance can be improved by a (theoretical) factor of 5.

However, the number of cardiac cycles is not limited to the numbers. Accordingly, more than 100 cardiac cycles can be provided, for example to compensate for relatively prominent outliers. However, data points from 10 to 40 cardiac cycles can also be evaluated, for example to enable rapid adaptation to a changed physiological state.

The determination of at least one amplitude change may furthermore be manually adjusted, for example, to extend or limit a fixed period or time interval. Preferably, the control unit is configured, in a coupled state with a display, to output to the display a signal for the representation of successive cardiac cycles detected from the ECG signal for mutually corresponding time points of the determined at least one amplitude change, and an adjustable temporal range indication, which marks the range of the evaluated data points. The evaluation unit may further advantageously be configured to receive an adjustment signal from or via the coupled display and to determine the at least one amplitude change for successive cardiac cycles in the adjusted relative time range, when the time range is or has been adjusted.

Thus, for example, an overlap of the respective cardiac cycles or a chronologically determined selection of the respective cardiac cycles with the currently determined at least one amplitude change can be shown on a display in a graphical representation, wherein a time window comprises the current time interval for evaluating the corresponding data points. By adjusting the time window, e.g., by shifting the limit values on a horizontal axis, the time interval can be shifted and/or lengthened or shortened, depending on the requirements of the displayed cardiac cycles with regard to a cardiac cycle phase that is relevant for at least one amplitude change and is being displayed. This provides the user with a certain flexibility and, as a result, even intuitive operation to optimize the at least one amplitude change.

Also for the temporal evaluation of the data points, the ECG signal may comprise at least a first measurement signal from a first ECG lead and a second measurement signal from a second ECG lead, wherein the first and second ECG leads are spatially separated from each other and wherein the evaluation unit is configured to determine the at least one amplitude change based on averaging or addition of the data points for the at least two measurement signals.

The data points from the two measurement signals may, for example, together form a value such that the data points are averaged both temporally and spatially. For example, at least one of the ECG leads can be configured as a transesophageal ECG lead and a corresponding probe or sensor. This has the advantage that the distance to a potential interfering signal, for example in case of heart stimulation, and thus the useful signal are improved accordingly.

A temporal averaging and a spatial addition of the data points may also be provided. In this case, for example, data points from at least two measurement signals from spatially separated ECG leads may be added or summed for the respective time point and the added data points can then be averaged for two or more cardiac cycles. In this way, the relationship or ratio between the useful signal and the interfering signal as well as the stability of a trigger signal are even further improved. Although a temporal averaging or a spatial addition individually already enables a significant improvement of the useful signal, the combination of the spatial and temporal evaluation is hence particularly advantageous to reduce potential interfering signals even further and to enable a more accurate signal-optimized circulatory support of the patient.

Preferably, the evaluation unit is furthermore configured to multiply the respective data points or the evaluated data points, in particular to exponentiate them, preferably by a factor or exponent of greater than 1.3. Preferably, the factor is 1.3 to about 5.0 or 1.3 to 3.0 or 1.3 to 2.0.

In this way, the data points or the individual measurement signals are further improved, wherein higher measurement values in comparison to lower measurement values are emphasized and a potential interfering signal can thus be reduced. The factor or exponent can be dependent on both a detection frequency and a number of detected and evaluated cardiac cycles. The exponentiation in combination with the spatial and/or temporal evaluation may hence result in a further improvement of the useful signal and hence further facilitate the determining of the amplitude change to provide a more stable extracorporeal circulatory support of the patient.

The above-mentioned aspects are furthermore achieved by a system for the extracorporeal circulatory support of a patient. Accordingly, the system comprises a device for extracorporeal circulatory support, comprising a blood pump, which is fluidically connectable to a venous patient access and an arterial patient access and is adapted to provide a blood flow from the venous patient access to the arterial patient access, an interface for receiving an ECG signal from the patient, and a control unit as described in the above, which is communicatively coupled to the device and wherein the control signal is a control signal for setting or adjusting the blood pump.

Preferably, the system also includes an ECG device which is communicatively connected to the interface.

The control unit may, for example, be formed as part of an ECG device or be integrated into it and thus be coupled to the system as an independent unit. The ECG device can thus be communicatively connected to the interface of the system. This also allows the system to be used independently of the presence of other components. Preferably, the ECG device is integrated in a single housing of the system, for example in a sensor box in the form of an ECG card or an ECG module. Alternatively, however, the control unit can also be configured to receive an external ECG signal from the supported patient, for example from a heart monitor located or arranged outside of the system. Thereby, the system may be formed even more compact.

The control unit can also be housed in a console with a user interface for entering and reading system settings, especially parameters of the blood pump and/or ECG device. For example, the console may include a touch screen and/or a display with a keyboard that can be operated by a user. The control unit operates, actuates, controls, regulates and monitors the blood pump and enables the blood pump to be synchronized with the cardiac cycle of the respective patient.

For example, the control unit can record the received ECG signal and the heart rate, wherein the display shows the current ECG signal graphically and the current or averaged trigger frequency and/or trigger stability numerically. Furthermore, characteristic features of the ECG signal or the respective cardiac cycle can be emphasized or marked in the graphic display, such that, for example, a trigger signal determined as an amplitude change in a QRS signal in the form of an R-wave can be marked in the ECG signal or in the current cardiac cycle. Furthermore, further settings such as the time interval between multiple amplitude changes or trigger signals or the heart rate can be represented in the ECG signal, such that a user can monitor the control and regulation of the blood pump with regard to the physiological condition of the patient.

The interface can, e.g., be configured as a sensor box, which may be connected via connectors to various sensors, such as pressure sensors of an extracorporeal circulatory support device, and to an ECG device.

The above-mentioned aspects are further achieved by a method for controlling/regulating of an extracorporeal circulatory support device. The method comprises at least the following:
receiving a measurement of an ECG signal of a supported patient over a predefined period of time, the ECG signal comprising multiple data points for each time point within a cardiac cycle,
evaluating the data points for at least one time point, wherein the evaluation is performed with spatially and/or temporally resolved and wherein at least one amplitude change within the cardiac cycle is determined from the evaluated data points, and
setting of a control and/or regulating signal for the extracorporeal circulatory support at a predefined time point after the at least one amplitude change.

Preferably the at least one determined or specific amplitude change is characteristic for a P-wave or R-wave. Thus, for example, a trigger signal can be output on the basis of a certain R-wave, wherein the stability of the trigger signal is considerably improved by the evaluation of the data points in spatial and/or temporal respect.

According to the method it may be accordingly provided that the ECG signal comprises at least a first measurement signal from a first ECG lead and a second measurement signal from a second ECG lead, wherein the first and second ECG leads are spatially separated from each other and wherein the data points are evaluated spatially and the at least one amplitude change is determined based on an addition or averaging of the measurement signals. Preferably, the ECG signal comprises a measurement signal of a transthoracic ECG lead and/or a transesophageal lead.

For improved monitoring of the temporal trigger stability, at least one amplitude change may be determined for at least two cardiac cycles and a time interval and/or a frequency of the amplitude changes may be determined, wherein a signal characterizing the time interval and/or the frequency is output. For example, the signal can include a graphical representation, wherein trigger signals are marked on the basis of the determined amplitude changes in the cardiac cycles or under the corresponding respective time point.

Furthermore, it may be provided that the at least one amplitude change is determined continuously during or for each successive cardiac cycle detected from the ECG signal. Thus, the determination of the at least one amplitude change can be immediately adapted to a current change in the physiological state of the patient.

Finally, a temporal evaluation of the data points may be provided. Accordingly, any time point of each cardiac cycle may be used, wherein the data points of successive cardiac cycles that correspond in time are temporally evaluated and the at least one amplitude change is determined based on averaging or addition of the data points from at least two cardiac cycles recorded for at least one time point (that is equally spaced in time relative to the reference point).

Preferably the at least one amplitude change is determined based on averaging the data points from at least 10, e.g., 10 to 100 cardiac cycles, preferably at least 40, e.g., between 40 and 80 cardiac cycles or between 10 and 40 cardiac cycles.

Due to the temporal averaging or addition, individual outliers, which, for example, do not lie in a relevant cardiac cycle range and are therefore not characteristic of a particular or specific cardiac cycle phase, do not impair the determination of the amplitude change, since the value of the corresponding data point is comparatively small for other cardiac cycles. In this manner it can be monitored in real time whether the determined amplitude change is within the intended or predefined range and whether a trigger signal is stable.

The determination of at least one amplitude change may furthermore be manually adjusted, for example, to extend or limit a specified time period or time interval. Accordingly, the successive cardiac cycles detected from the ECG signal for matching time points, each of which is relative to the same reference point, the at least one amplitude change, and an adjustable time range indication which characterizes the range of the evaluated data points are shown on a display, wherein, upon receiving an adjustment signal from the coupled display, the at least one amplitude change is determined in the adjusted relative time range related to the same reference point for successive cardiac cycles.

In addition to a temporal averaging, it may further be provided that the ECG signal comprises at least a first measurement signal from a first ECG lead and a second measurement signal from a second ECG lead, wherein the first and second ECG leads are spatially separated from each other and wherein the at least one amplitude change is determined based on averaging or addition of the data points for the at least two measurement signals.

Further advantages as well as possible embodiments and further extensions of the methods have already been described in detail with regard to the control unit described in the above, such that a repeated description of the corresponding aspects is not provided in order to avoid redundancies. However, the corresponding disclosure contents continue to apply to the method.

The above-mentioned aspects are further achieved by a method for monitoring a temporal trigger stability of an extracorporeal circulatory support. The method comprises at least the following:
receiving a measurement of an ECG signal of a supported patient over a predefined period of time, wherein the ECG signal comprises multiple data points for each time point within a cardiac cycle,
evaluating the data points for at least one time point, wherein the evaluation is performed spatially and/or temporally and wherein at least one amplitude change within the cardiac cycle is determined from the evaluated data points, wherein the at least one determined amplitude change is preferably characteristic of a P-wave or R-wave, and wherein at least one amplitude change is determined for at least two cardiac cycles,
determining a time interval and/or frequency of the amplitude changes, and
outputting a signal, when the time interval and/or frequency of the amplitude changes exceeds a predefined threshold.

The determining of the time interval and/or frequency of the amplitude changes enables that a temporal stability is provided, i.e., whether a trigger signal with similar time intervals and at the correct time points with respect to the respective cardiac cycle phases is output based on the determined amplitude changes. For example, a small deviation may be ignored, but a deviation over a predefined percentage of the time interval, such as when a tolerance range between 10 and 15 percent of the average time interval is exceeded, may result in the outputting of a signal.

The signal may include an audible warning signal as well as a visual mark or warning on a display, for example, in a section for a time line of the outputted trigger signals.

### BRIEF DESCRIPTION OF THE DRAWINGS

Embodiments of the present disclosure are explained in more detail in the following description of the Figures.

FIG. 1 shows the course of an electrocardiogram with a sinus rhythm for a plurality of transthoracic ECG leads and two transesophageal ECG leads.

FIGS. 2A to 2E show an electrocardiographic course of spatially separated ECG leads without stimulation and with stimulation of the heart by an implanted pacemaker.

FIG. 3 is a schematic representation of a control unit.

FIG. 4 shows an electrocardiographic course of two spatially separated ECG leads for a predefined period of time.

FIG. 5 shows the determining of multiple amplitude changes based on a spatial evaluation of the data points shown in FIG. 4.

FIG. 6 shows the outputting of control signals based on the amplitude changes determined in FIG. 5.

FIGS. 7A and 7B show alternative spatial evaluations and graphical representations of the data points.

FIGS. 8A through 8C show the determining of amplitude changes based on a temporal evaluation for a different number of cardiac cycles and a predefined time interval.

FIG. 9 shows a monitoring and graphical adjustment possibility of the time interval for determining the amplitude change.

### DETAILED DESCRIPTION

In the following, embodiments will be explained in more detail with reference to the accompanying Figures. In the Figures, corresponding, similar, or like elements are denoted by identical reference numerals and repeated description thereof may be omitted in order to avoid redundancies.

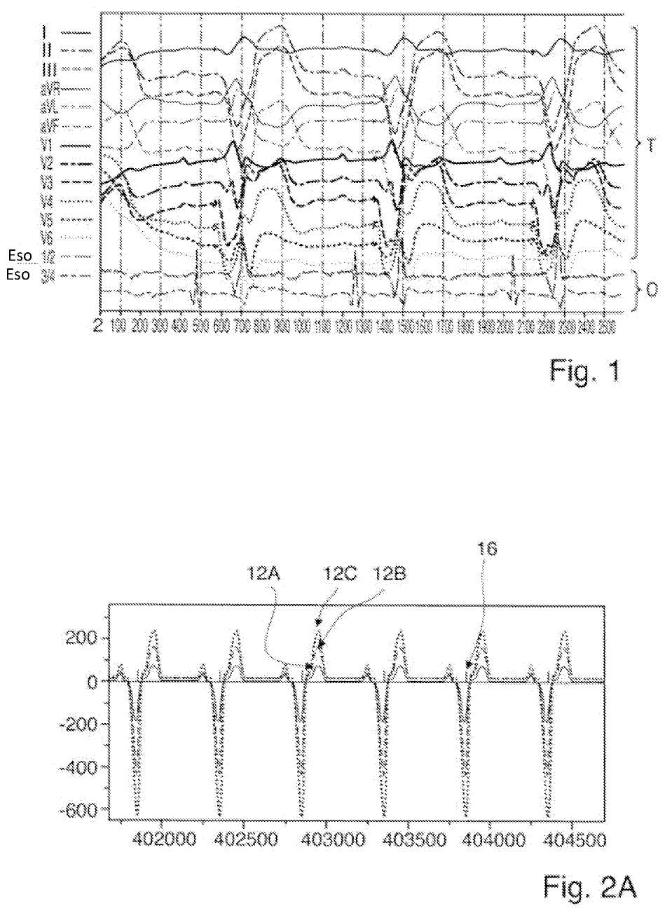

FIG. 1 shows the progression or course of an electrocardiogram with a sinus rhythm for a plurality of transthoracic (T) ECG leads and two transesophageal (O) ECG leads, wherein according to this example ECG signals are shown from a patient with a grade III AV block and with right atrial synchronous bipolar right ventricular stimulation in DDD mode of an implanted cardioverter/defibrillator (ICD). However, the course shown in this Figure should only be regarded as exemplary. Other aspects determined in the course of ECG signal recording and characteristic for other heart diseases or therapies can also be recorded or detected accordingly. In other words, the example shown here is not limited to the specific pathophysiological condition and/or therapy.

The measurement signals of the ECG leads, which are recorded and displayed in FIG. 1 for a predefined period of time, include measurement signals from the transthoracic ECG leads I, II, III, aVR, aVL, aVF, V1, V2, V3, V4, V5, and V6 and from bipolar transesophageal ECG leads Oeso 12 and Oeso 34. The number and type of leads, however, is not to be regarded as limiting. In principle, any selection of ECG leads can be used to determine at least one amplitude change. Thus, a spatially separated detection of measurement signals can be performed, both within one anatomical area and for different anatomical areas.

Accordingly, the measurement signals can be processed and evaluated, for example, to enable a spatial and/or temporal analysis or evaluation.

The spatial and/or temporal evaluation of data points of an ECG signal has the advantage that the ratio of the useful signal to the interfering signal can be improved. Thereby an amplitude change within a cardiac cycle can be determined more accurately. Interfering signals can, for example, occur as a result of stimulation of the heart or for pathophysiological reasons or even spontaneously, such that the regularity or stability of the ECG signal is reduced and the determination of an amplitude change, for example an R-wave, is impaired.

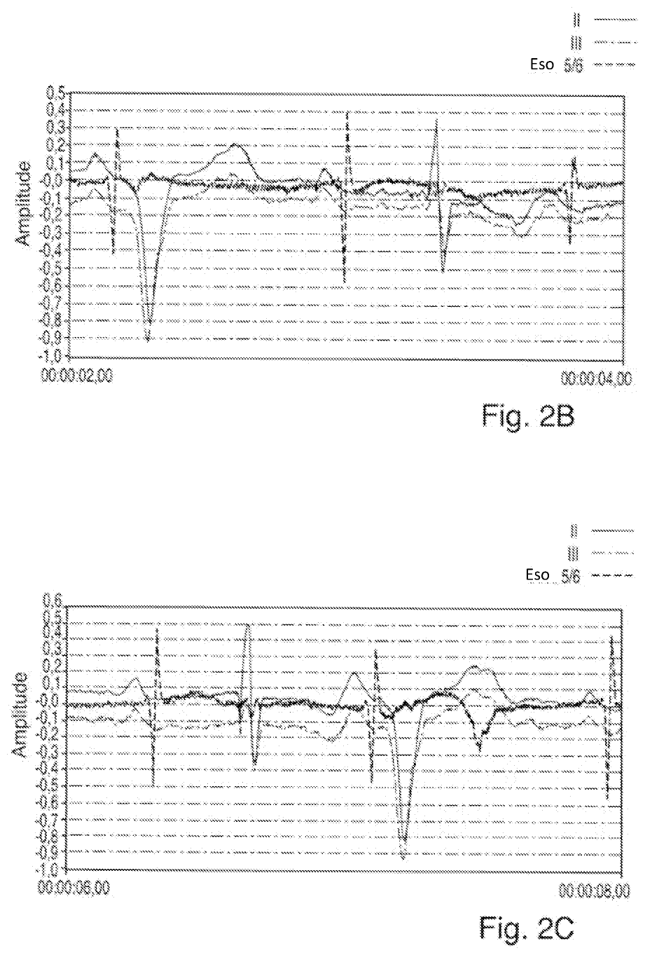

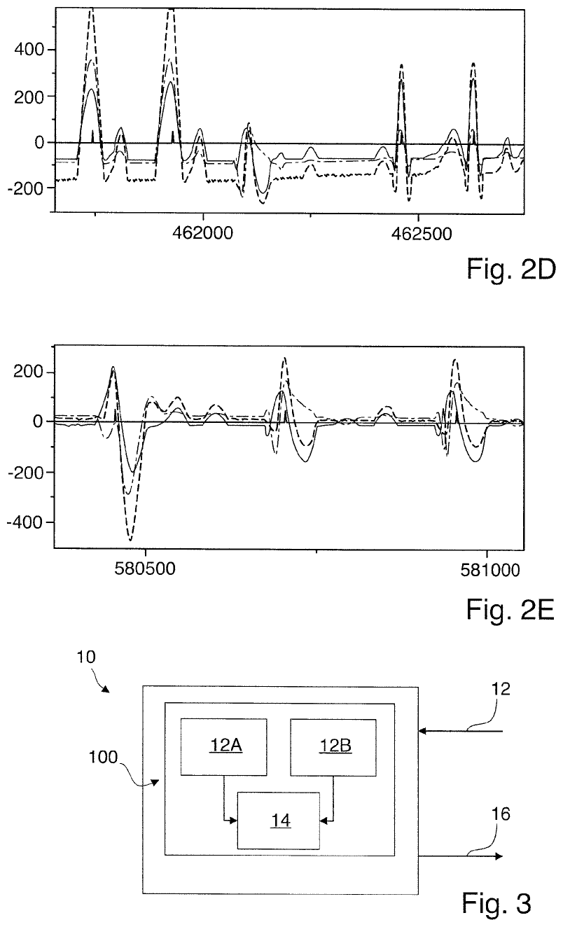

An example of a corresponding ECG signal with different complex heart actions is shown in FIGS. 2A to 2E in a non-stimulated state and a stimulated state.

FIG. 2A accordingly shows an electrocardiographic course of a first 12A and second 12B measurement signal from two spatially separated ECG leads and a corresponding sum signal 12C, wherein ECG leads II and III are transthoracic ECG leads. In the course six amplitude changes are determined by detecting R-peaks 16 or R-waves, wherein the course depicts the corresponding measurement signals during atrial-triggered ventricular stimulation from left to right. In this course, a relatively stable determination of the R-wave 16 is possible and the measurement signals 12A, 12B show no significant fluctuations or anomalies. FIG. 2B shows an electrocardiographic course of three spatially separated ECG leads, wherein ECG leads II and III are transthoracic ECG leads and ECG lead Oeso 5/6 is a left-atrial bipolar transesophageal ECG lead. In the course, two amplitude changes are determined by detecting R-waves, wherein the course of the signals is represented from left to right in case of inhibition of bipolar right ventricular stimulation, in case of ventricular extrasystole and in case of a narrow QRS complex. Accordingly, fluctuations due to different cardiac arrhythmias are present, which render it difficult to determine a change in amplitude, as is the case with an R-wave.

In FIG. 2C, corresponding successive amplitude changes are also determined, wherein the course depicts the corresponding measurement signals from left to right in the case of bipolar right ventricular fusion stimulation and in the case of ventricular extrasystole with inhibition of bipolar right ventricular stimulation. Thus, it can be seen that a variation of the measurement signals can occur, which can lead to instability of a trigger signal, if the amplitude change cannot be determined with sufficient accuracy over time.

In FIG. 2D, two right ventricular stimulated heart actions followed by one ventricular extrasystole and two spontaneous heart actions are shown from left to right while in FIG. 2E two QRS morphologies are shown. Accordingly, depending on the patient, the heart disease and stimulation, different fluctuations can be recorded from the measurement signals.

A schematic representation of a control unit 10 is shown in FIG. 3. In this embodiment, the control unit 10 is configured to receive an ECG signal 12 for two different ECG leads, wherein an evaluation unit 100 in the control unit 10 evaluates the corresponding data points for two different measurement signals 12A, 128. Thus, for example, a spatial evaluation can be performed to facilitate the determining of an amplitude change 14 and to improve the ratio of the useful signal to the interfering signal.

The control unit 10 is configured or formed as an ECG module, such that no particular coupling is required to receive the ECG signal. However, the ECG module can include an interface (not shown), which enables a communicative coupling with an extracorporeal circulatory support system or an extracorporeal circulatory support device, such that it can be controlled or regulated accordingly by the control unit 10.

The control unit 10 is also configured to output a control signal 16 based on the determined amplitude change. For example, one or more amplitude changes 14 can be determined, which are characteristic for an R-wave in the respective cardiac cycle. The control signal 16 can then be output as an R-trigger signal and, for example, may be used to operate or control a blood pump with a latency period to provide an improved perfusion of the patient's coronary arteries.

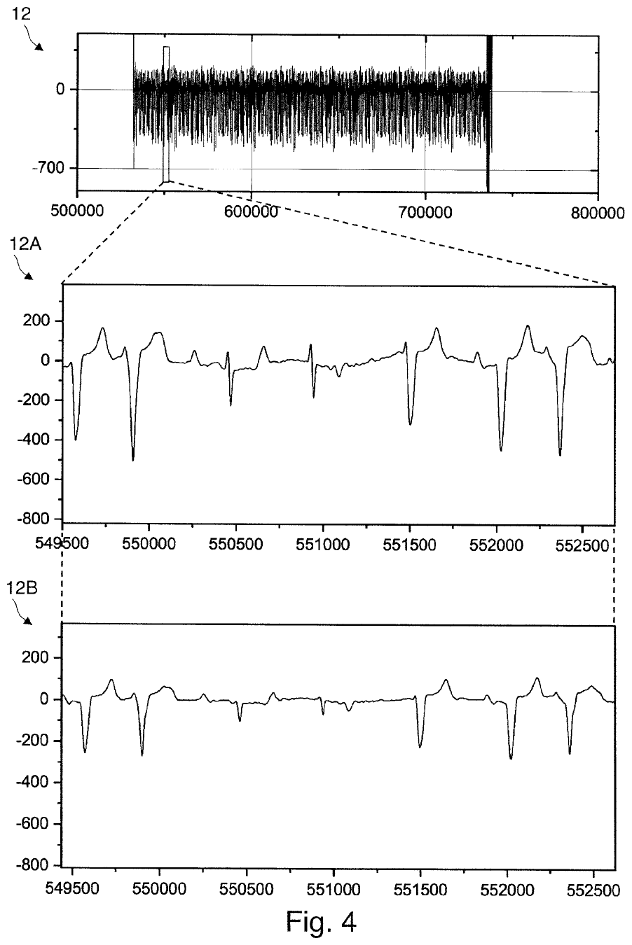

An example of a spatial evaluation is shown in FIGS. 4 to 6. In FIG. 4 (top) an ECG signal 12 is recorded and shown. For a predefined period of time, first measurement signals 12A from a first ECG lead (middle) and second measurement signals 12B from a second ECG lead (bottom) were recorded and received by the control unit or the evaluation unit. In this example, the ECG leads are spatially separated from each other and correspond to ECG leads II (middle) and III (bottom) of FIGS. 1 and 2.

In this example, the ECG signal 12 is from a patient with coronary artery disease with a left ventricular ejection fraction of 65%, with sinus rhythm, higher grade AV block and intermittent bipolar right ventricular stimulation in a VVIR mode of an implanted pacemaker. FIG. 4 shows a total of seven cardiac actions of ECG leads II and III in the predefined time period, wherein the y-axis shows the amplitude of the measurement signal 12A, 12B and the x-axis shows the time course of the ECG signal 12 with a sampling rate of 500 Hz, so that an interval of “500” corresponds to 1000 ms.

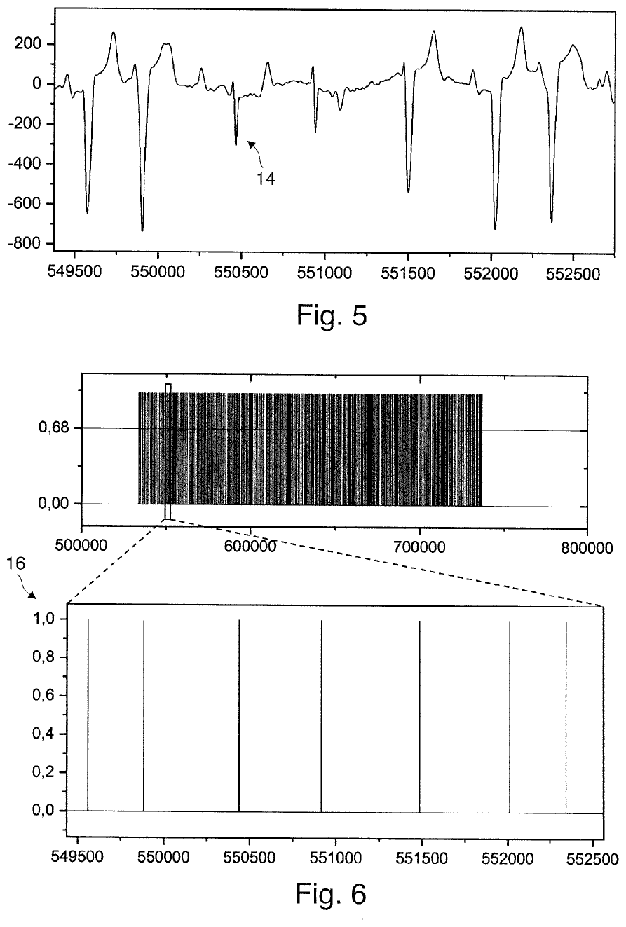

From the shown measurement signals 12A and 126 it follows that the useful signals are different between the different heart actions and for the different anatomical areas and can vary accordingly, not only in amplitude size, but also in their temporal distribution. In the evaluation unit of the control unit, however, the measurement signals can be evaluated spatially or with spatial resolution and, for example, may be added together, as shown in FIG. 5. By adding the measuring signals 12A, 12B, the ratio of the useful signal to the interfering signal can thus be improved by a factor of 1.4, such that the determination of the amplitude change 14 can be considerably facilitated and provided more accurately. This is shown, for example, by the amplitude improvement for the third and fourth heart action in FIG. 5 with respect to the corresponding amplitudes in FIG. 4.

The improvement of the amplitudes allows to improve the output of the control signal 16, since the amplitude change can be accurately determined and thus, for example, a maximum slope or a maximum of the amplitude can be determined more accurately. Accordingly, the amplitude change can serve as trigger signal 16 for an extracorporeal circulatory support, wherein a temporal stability of the trigger signal 16 is ensured.

This follows, e.g., from FIG. 6, wherein the temporal distance between two respective trigger signals 16 does not show any severe irregularities and trigger signals 16 can thus be provided or output for control/regulation of an extracorporeal circulatory support in corresponding cardiac cycle phases. In this example, the control signals 16 and trigger signals 16 are output on the basis of determined R-peaks. However, these signals 16 can also be output with a corresponding latency time on the basis of determined P-waves or other characteristic amplitude changes of the ECG signal.

The improved temporal trigger stability based on the spatial evaluation of the data points can thus be particularly advantageous for the precise control of an extracorporeal circulatory support, wherein interfering signals can be suppressed or corrected. For example, interfering signals resulting from intermittent stimulation, such as bipolar right ventricular stimulation, can be suppressed or corrected in a patient with an implanted pacemaker with heart failure and coronary artery disease, but with normal left ventricular pumping function.

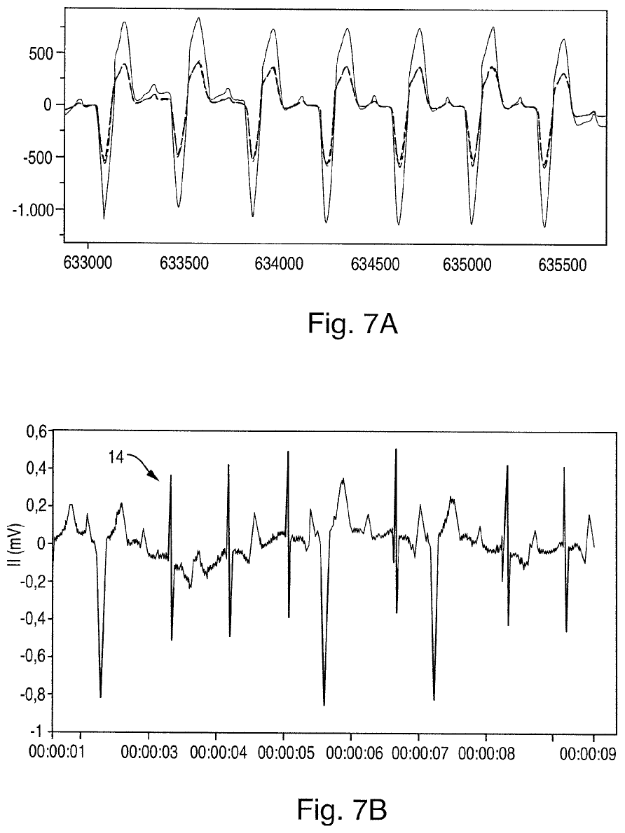

FIGS. 7A and 7B show alternative spatial evaluations and graphical representations of the data points. Accordingly, the data points can be spatially evaluated for the entire period of time as shown in FIG. 7A and displayed by means of an overlap and color coding on a display coupled to the control unit, such that the improvement of the useful signal and also the temporal stability of the determined amplitude changes can be easily monitored.

The evaluation of the data points can also only be performed for a specific time interval or for a specific time range of a cardiac cycle phase, as shown in FIG. 7B for an alternative data set.

Accordingly, a spatial evaluation or addition of the measurement signals is carried out, for example, only for the R-wave, which can be detected on the basis of a slope of a first measurement signal. The spatially evaluated data points can be represented as an extension of the data points of the first measurement signal for a particular time range of a cardiac cycle phase, wherein this range is defined, for example, by exceeding a threshold value of the evaluated data points.

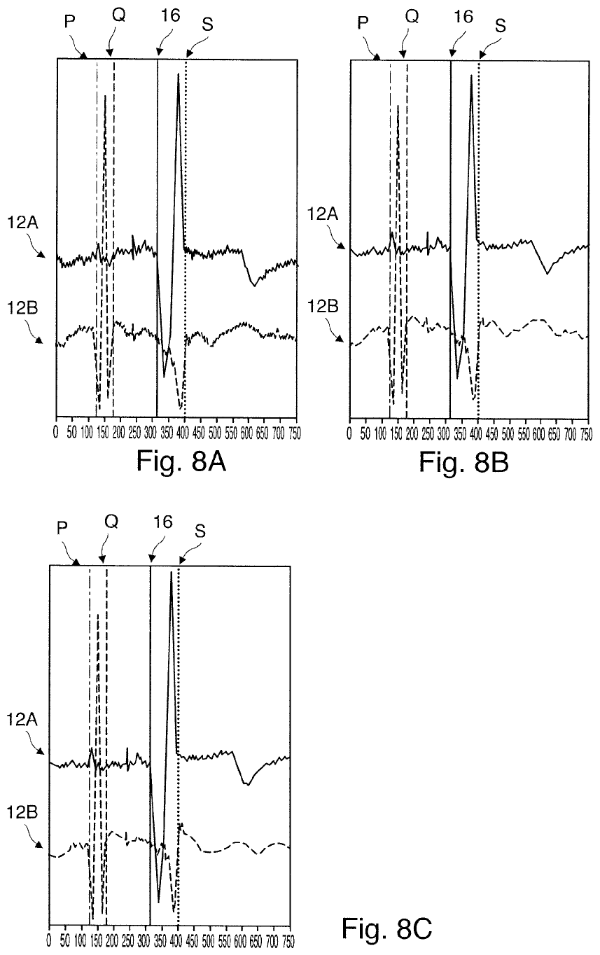

A temporal evaluation of the data points may be provided to determine amplitude changes, as shown in FIGS. 8A to 8C. In this exemplary embodiment, each time point forms a relative time point of each cardiac cycle. The evaluation unit is configured to evaluate the data points temporally and to determine the at least one amplitude change based on averaging the data points for at least one corresponding time point from at least two cardiac cycles and that has an identical temporal distance to the same reference point in the at least two cardiac cycles, e.g., for the at least two cardiac cycles always the maximum of a signal, e.g. the R-wave, as shown in FIG. 8A.

The data points are averaged accordingly for each of the time points corresponding in time in the cardiac cycles. Alternatively, however, an addition can be provided. The change in amplitude is determined by the course within a particular time interval, in the present example of a detected QRS complex, where P marks the beginning of the P-wave, Q the beginning of the Q-wave and S the end of the S-wave in FIGS. 8A to 8C. For example, the data points can be evaluated in time using the following formula:

$$\overline{{X}_{j}} = \frac{1}{n} {\sum }_{i = 1}^{n} Xij$$

where n points are summed and where j is a single data point within the time period for a particular time point corresponding to each of the n cardiac cycles and i is a respective cardiac cycle. Accordingly, an average value is calculated or formed for the corresponding n data points. A time point in the n cardiac cycles is, for example, in each cardiac cycle after a period of y₁ (μs) before or after the occurrence of the reference point (e.g. the maximum of a signal in the ECG, e.g. the R-wave) in the respective cardiac cycle. For each cardiac cycle a data point at time y₁ is determined and an average value for these data points at time y₁ in all cardiac cycles is formed. The same procedure is performed at time y₂ to yₙ.

The amplitude change serves furthermore for the output of a corresponding control signal 16 or a trigger signal 16 and is marked accordingly within the QRS complex.

Due to the temporal averaging between multiple cardiac cycles for each time point that corresponds temporally in the cardiac cycles, the different cardiac cycle phases of successive cardiac cycles and in particular the course of these cardiac cycle phases can be compared with each other, so that data points for the same time point corresponding in time to the selected reference point, but for different successive cardiac cycles, represent a useful signal for the same respective cardiac cycle phase. The temporal averaging thus allows that individual outliers, which for example do not lie in a relevant cardiac cycle range and are therefore not characteristic for a particular cardiac cycle phase, nevertheless do not impair the determination of the amplitude change, since the value of the corresponding data point is relatively small for other cardiac cycles.

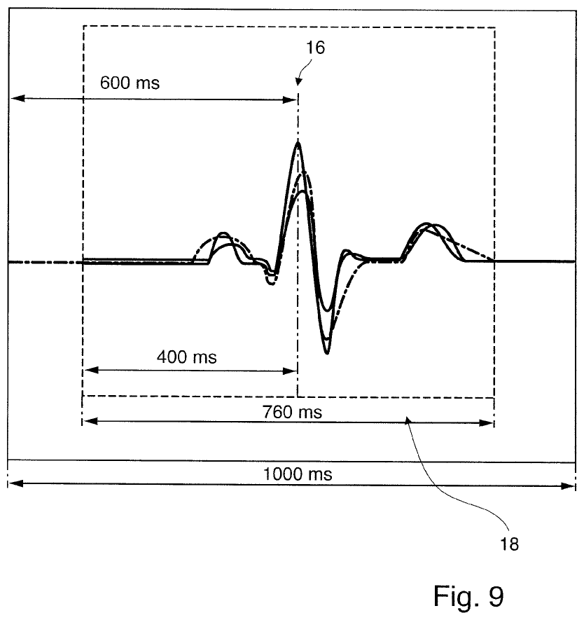

An increase in the number of compared cardiac cycles can further improve the temporal stability of the trigger signal 16, for example in the case of a regular heart rhythm, as can be seen, for example, from FIG. 8B with ten cardiac cycles and 8C with 65 cardiac cycles. The data points and the measurement signals in the present example were not only increased in number, but also exponentiated, such that the data points with a low measurement signal value are less prominent. This is illustrated by the fact that the respective curves run even less jagged, i.e., with fewer deflections, if the number of cardiac cycles is increased at the same time, so that interfering signals can be at least partially suppressed. FIG. 9 shows a monitoring and graphic adaptation or adjustment possibility of the time interval 18 for determining the amplitude change.

With a temporal averaging, a particular or predefined trigger point can be required for the number n of heart cycles or heart actions, which is preferably an R-trigger 16. The determination of the at least one amplitude change can further be adjusted manually, for example to extend or limit a fixed or selected period or time interval. Preferably, the control unit, in a coupled state with a display, is hence configured to output to the display a signal representing successive cardiac cycles detected from the ECG signal for the respective corresponding time points, the determined at least one amplitude change, and an adjustable time range indication, which marks the range of the evaluated data points. The evaluation unit is further advantageously configured to receive an adjustment signal from or via the coupled display and to determine the at least one amplitude change in the adjusted relative time range for successive cardiac cycles upon an adjustment of the time range.

Accordingly, an overlap of the current cardiac cycle with the two last cardiac cycles is shown in a graphic representation as an example. Also, the graphic representation shows the currently determined, at least one amplitude change and a time marker for the current output of the control and regulation signal 16 or the trigger signal 16 on a display coupled to the control unit, wherein a time window comprises the current time interval 18 for evaluating the corresponding data points. In other words, the time window forms a monitoring period for a sampling complex, wherein the cardiac cycles are preferably completely within the time window in order to record a complete data set within the time interval 18. In FIG. 9 a schematic representation of the cardiac cycles is shown having a deliberate morphology.

The shown time intervals are also only exemplary, but can also be preset as predefined values. Preferably, the time window is selected or set in such a way that at least the current cardiac cycle is shown starting from a morphologically and/or physiologically predefined reference point and preferably also up to a corresponding reference point. For example, the time window can thus represent a time interval 18, which at least represents the current cardiac cycle from the end of the preceding T-wave to the end of the current T-wave.

By adjusting the time window, e.g., by shifting the limits on a horizontal axis, the time interval 18 can be shifted and/or lengthened or shortened, depending on how the displayed cardiac cycles require a cardiac cycle phase relevant for the at least one amplitude change. This provides the user with a certain flexibility and even intuitive operation to optimize the at least one amplitude change. Thus, when adjusting or shifting the time window, the position of the trigger signal in the window can also be shifted (not shown). In the present case, three successive cardiac cycles, i.e., the current and the last two cardiac cycles, are displayed in an overlapping manner, but it is also possible that only two or more cardiac cycles are provided to determine the amplitude change, for example 10 or 65, as described above.

Where applicable, all the individual features depicted in the exemplary embodiments may be combined and/or exchanged without leaving the scope of the invention.

### LIST OF REFERENCE NUMERALS

10 Control unit
12 ECG signal
12A First measuring signal
12B Second measuring signal
12C Sum signal
14 Amplitude change
16 Control signal or trigger signal
18 Time interval
100 Evaluation unit
O Transesophageal ECG lead
P Start of P-wave
Q Start of Q-wave
S End of S-wave
T Transthoracic ECG lead

1-32. (canceled)

33. A control unit for an extracorporeal circulatory support, the control unit configured to:
receive a measurement of an ECG signal of a supported patient over a predefined period of time, wherein the ECG signal comprises multiple data points for each time point within a cardiac cycle;
wherein the control unit comprises an evaluation unit which is configured to evaluate the data points spatially and/or temporally for at least one time point and to determine at least one amplitude change within the cardiac cycle from the evaluated data points; and
wherein the control unit is further configured to output a control signal for the extracorporeal circulatory support at a predefined time point after the at least one amplitude change.

34. The control unit according to claim 33, wherein the evaluation unit is configured to evaluate the data points for a predefined time interval based on at least one cardiac cycle phase of the ECG signal and to determine the at least one amplitude change within the time interval.

35. The control unit according to claim 33, wherein the evaluation unit is configured to determine the at least one amplitude change based on data points for at least two time points.

36. The control unit according to claim 33, wherein the evaluation unit is configured to determine at least one specific amplitude change which is characteristic of a cardiac cycle phase.

37. The control unit according to claim 36, wherein the at least one specific amplitude change is characteristic of a P-wave or R-wave.

38. The control unit according to claim 33, wherein the ECG signal comprises at least a first measurement signal from a first ECG lead and a second measurement signal from a second ECG lead, wherein the first and second ECG leads are spatially separated from one another and wherein the evaluation unit is configured to spatially evaluate the data points and to determine the at least one amplitude change based on an addition or averaging of the measurement signals.

39. The control unit according to claim 33, wherein the ECG signal comprises a measurement signal of a transthoracic ECG lead and/or a transesophageal ECG lead and/or wherein the ECG signal is an ECG signal of a patient with cardiac stimulation.

40. The control unit according to claim 33, wherein the evaluation unit is configured to determine an amplitude change for at least two cardiac cycles and a time interval and/or a frequency of the amplitude changes, wherein the control unit is configured to output a signal characterizing the time interval and/or the frequency.

41. The control unit according to claim 33, wherein the evaluation unit is configured to determine the at least one amplitude change continuously at each successive cardiac cycle detected from the ECG signal.

42. The control unit according to claim 41, wherein the evaluation unit is configured to determine the at least one amplitude change in real time.

43. The control unit according to claim 33, wherein the evaluation unit is configured to evaluate the data points temporally and to determine the at least one amplitude change based on an addition or averaging of the data points for at least one time point corresponding in time in the at least two cardiac cycles.

44. The control unit according to claim 43, wherein the evaluation unit is configured to determine the at least one amplitude change based on averaging or addition of the data points from a number of cardiac cycles between 10 to 40 cardiac cycles.

45. The control unit according to claim 43, wherein the evaluation unit is configured to determine the at least one amplitude change based on averaging or addition of the data points from a number of cardiac cycles between 10 to 100 cardiac cycles.

46. The control unit according to claim 43, wherein the evaluation unit is configured to determine the at least one amplitude change based on averaging or addition of the data points from a number of cardiac cycles between 40 and 80 cardiac cycles.

47. The control unit according to claim 43, wherein the at least one time point corresponding in time between the at least two cardiac cycles is defined by identical temporal spacing from a reference point occurring in each of the at least two cardiac cycles.

48. The control unit according to claim 47, wherein the reference point is morphologically and/or physiologically predefined by a signal in the ECG.

49. The control unit according to claim 48, wherein the reference point is predefined by a maximum in the ECG signal.

50. The control unit according to claim 43, which in the coupled state with a display is arranged to output a signal for representation of:
successive cardiac cycles detected from the ECG signal for respective corresponding points in time;
the determined at least one amplitude change; and
an adjustable temporal range indication, which represents the range of the evaluated data points
to the display, wherein the evaluation unit is further configured to receive an adjustment signal from the coupled display and to determine the at least one amplitude change upon adjustment of the temporal range for successive cardiac cycles in an adjusted relative temporal range.

51. The control unit according to claim 43, wherein the ECG signal comprises at least a first measurement signal from a first ECG lead and a second measurement signal from a second ECG lead, wherein the first and second ECG leads are spatially separated from each other and wherein the evaluation unit is configured to determine the at least one amplitude change based on averaging or addition of the data points for the at least two measurement signals.

52. The control unit according to claim 33, wherein the evaluation unit is configured to exponentiate the respective data points or the evaluated data points.

53. The control unit according to claim 52, wherein the evaluation unit is configured to exponentiate the respective data points or the evaluated data points with an exponent of greater than 1.3.

54. The control unit according to claim 53, wherein the exponent is in a range of 1.3 to 5.0.

55. The control unit according to claim 54, wherein the exponent is in a range of 1.3 to 2.0.

56. A system for extracorporeal circulatory support of a patient, the system comprising:
a device for extracorporeal circulatory support, comprising a blood pump which is fluidically connectable to a venous patient access and an arterial patient access and is adapted to provide a blood flow from the venous patient access to the arterial patient access;
an interface for receiving an ECG signal from the patient; and
a control unit communicatively coupled to the device and configured to:
receive a measurement of the ECG signal of the patient over a predefined period of time, wherein the ECG signal comprises multiple data points for each time point within a cardiac cycle;
wherein the control unit comprises an evaluation unit which is configured to evaluate the data points spatially and/or temporally for at least one time point and to determine at least one amplitude change within the cardiac cycle from the evaluated data points; and
wherein the control unit is further configured to output a control signal for the extracorporeal circulatory support at a predefined time point after the at least one amplitude change, wherein the control signal is a control signal for setting the blood pump.

57. The system according to claim 56, further comprising an ECG device communicatively coupled to the interface.

58. A method for controlling an extracorporeal circulatory support, the method comprising:
receiving a measurement of an ECG signal of a supported patient over a predefined period of time, wherein the ECG signal comprises multiple data points for each time point within a cardiac cycle;
evaluating the data points for at least one time point, wherein the evaluation is performed spatially and/or temporally and wherein at least one amplitude change within the cardiac cycle is determined from the evaluated data points, and
setting of a control signal for the extracorporeal circulatory support at a predefined time point after the at least one amplitude change.

59. The method according to claim 58, wherein the at least one determined amplitude change is characteristic of a P-wave or R-wave.

60. The method according to claim 58, wherein the ECG signal comprises at least one first measurement signal from a first ECG lead and a second measurement signal from a second ECG lead, wherein the first and second ECG leads are spatially separated from each other and wherein the data points are spatially evaluated and the at least one amplitude change is determined based on an addition and/or averaging of the measurement signals.

61. The method according to claim 58, wherein the ECG signal comprises a measurement signal of a transthoracic ECG lead and/or a transesophageal lead.

62. The method according to claim 58, wherein the at least one amplitude change is determined for at least two cardiac cycles and a time interval and/or a frequency of the amplitude changes is determined, wherein a signal representing the time interval and/or the frequency is output.

63. The method according to claim 58, wherein the at least one amplitude change is determined continuously for each successive cardiac cycle detected from the ECG signal.

64. The method according to claim 58, wherein each time point is selected at a temporal distance from the reference point that is equal for each cardiac cycle and wherein the data points are evaluated temporally and the at least one amplitude change is determined based on an averaging or addition of the data points for at least one time point from at least two cardiac cycles corresponding temporally relative to a reference point.

65. The method according to claim 64, wherein the at least one amplitude change is determined based on an averaging or addition of the data points from a number of cardiac cycles between 10 to 100 cardiac cycles.

66. The method according to claim 64, wherein the at least one amplitude change is determined based on an averaging or addition of the data points from a number of cardiac cycles between 10 to 40 cardiac cycles.

67. The method according to claim 64, wherein the at least one amplitude change is determined based on an averaging or addition of the data points from a number of cardiac cycles between 40 and 80 cardiac cycles.

68. The method according to claim 64, wherein the time point for each cardiac cycle is constant in time relative to a reference point.

69. The method according to claim 68, wherein the reference point is predefined morphologically and/or physiologically by the ECG signal.

70. The method according to claim 64, wherein successive cardiac cycles detected from the ECG signal for time points corresponding in time relative to a reference point, the at least one amplitude change, and an adjustable temporal range indication, which represents the range of the evaluated data points, are shown on a display and wherein upon receiving an adjustment signal from the coupled display the at least one amplitude change is determined in the adjusted relative time range for successive cardiac cycles.

71. The method according to claim 58, wherein the ECG signal comprises at least a first measurement signal from a first ECG lead and a second measurement signal from a second ECG lead, wherein the first and second ECG leads are spatially separated from each other and wherein the at least one amplitude change is determined based on averaging or addition of the data points for the at least two measurement signals.

72. The method according to claim 58, wherein the respective data points or the evaluated data points are exponentiated.

73. The method according to claim 72, wherein the respective data points or the evaluated data points are exponentiated with an exponent greater than 1.3.

74. The method according to claim 73, wherein the exponent is in a range of 1.3 to 5.0.

75. The method according to claim 73, wherein the exponent is in a range of 1.3 to 2.0.

76. A method for monitoring a temporal trigger stability of an extracorporeal circulatory support, the method comprising:
receiving a measurement of an ECG signal of a supported patient over a predefined period of time, wherein the ECG signal comprises multiple data points for each time point within a cardiac cycle;
evaluating the data points for at least one time point, wherein the evaluation is performed spatially and/or temporally and wherein at least one amplitude change within the cardiac cycle is determined from the evaluated data points, wherein at least one amplitude change is determined for at least two cardiac cycles;
determining a time interval and/or frequency of the amplitude changes; and
outputting a signal when the time interval and/or frequency of the amplitude changes exceeds a predefined threshold.

77. The method of claim 76, wherein the at least one determined amplitude change is characteristic of a P-wave or R-wave.
